(19) United States
(12) Patent Application Publication
(10) Pub. No.: US 2025/0195914 A1
(43) Pub. Date: Jun. 19, 2025

(54) METHOD FOR ENHANCING ANTIBODY FORMATION FUNCTION BY USING BIO INFORMATIVE ENERGY LIGHT

(71) Applicant: BIOLIGHT CORP., Hanam-si (KR)

(72) Inventors: Mi Jung PARK, Seoul (KR); Won You LEE, Namyangju-si (KR); Sang-Ik YUN, Seoul (KR); Haelim JANG, Gunpo-si (KR)

(73) Assignee: BIOLIGHT CORP., Hanam-si (KR)

(21) Appl. No.: 19/071,022

(22) Filed: Mar. 5, 2025

Related U.S. Application Data
(63) Continuation of application No. PCT/KR2023/012876, filed on Aug. 30, 2023.

(30) Foreign Application Priority Data
Sep. 5, 2022 (KR) 10-2022-0112437

(51) Int. Cl. A61N 5/06

(52) U.S. Cl. CPC A61N 5/0613; A61N 2005/0632; A61N 2005/0665; A61N 2005/0667

(57) ABSTRACT

To implement the objects described above, a method for enhancing an antibody-forming function using bio-informative energy light according to various embodiments of the present invention is disclosed. The method includes administering a vaccine to a mammal, and irradiating the vaccinated mammal with bio-informative energy light, wherein the bio-informative energy light has an intensity of 10⁻¹⁸ to 10⁻¹³ W/cm².

### CROSS-REFERENCE TO RELATED PATENT APPLICATION

This application is a Continuation of International Application No. PCT/KR2023/012876, filed on Aug. 30, 2023, which claims the benefit of Korean Patent Application No. 10⁻²⁰²²-0112437, filed on Sep. 5, 2022, in the Korean Intellectual Property Office, the disclosure of which is incorporated herein in its entirety by reference.

### TECHNICAL FIELD

The present invention relates to a method for improving the immune function of a living organism, and more particularly, to a method for enhancing an antibody-forming function using bio-informative energy light.

### BACKGROUND ART

Bio-informative energy is light or energy that is polychromatic in the visible light spectrum and whose intensity is so weak that it is equivalent to 1/500,000 of the brightness of a typical fluorescent light. Because this bio-informative energy is at least 1,000 times weaker than bioluminescence, it is highly efficient and safe. The possibility of bio-informative energy affecting living organisms was first raised in the academic world in the 1930s, and later, the German photobiophysicist Popp published experimental results showing that information exchange between cells is achieved through bio-informative energy. Based on this background, the safety and usefulness of a bio-informative energy generator has been confirmed by applying it to living organisms through many years of research.

Bio-informative energy is very weak in intensity and is called ultra weak photon emission or emission (biophotons). The phenomenon of biophoton generation is related to reactive oxygen species (ROSs) generated during the normal metabolic processes of living organisms. These ROSs are formed as natural byproducts of normal oxygen metabolism and play important roles in cell signaling and homeostasis.

This bio-informative energy can activate the metabolism of living organisms and enhance their immune capabilities. For example, bio-informative energy generated by a bio-informative energy generator may be radiated to living organisms, and accordingly, it can provide various effects such as enhancing the immunity and anti-aging/anti-oxidation abilities of living organisms, and shortening growth and shipping periods. Korean Unexamined Patent Publication No. 10⁻²⁰¹⁹-0127223 discloses a method for enhancing the immunity of shrimp by light irradiation.

Meanwhile, today, highly contagious viruses such as influenza and novel coronaviruses cause various diseases and threaten human life. Viruses are so small that they can easily spread through animal feces, water, air, etc., depending on the type. When a contagious virus appears in a densely populated area, it can spread throughout a city in an instant. The development of vaccines is essential to prevent the spread of such viruses. A vaccine is made by weakening the power of viruses causing a disease or using a part of the viruses, and when one is administered to a person, antibodies that have an immune effect are produced.

However, there is concern that even vaccine users may continue to be exposed to various viral infections due to insufficient antibody production, and there is concern that vaccination may cause side effects. Accordingly, the industry may be required to conduct research and development on a method to maximize the antibody-forming function of vaccinated users while simultaneously minimizing the side effects of vaccination using bio-informative energy light.

### DISCLOSURE

### Technical Problem

The problem to be solved by the present invention is to address the aforementioned issues by providing a method for enhancing the antibody-forming function using bio-informative energy light.

The problems to be solved by the present invention are not limited to those mentioned above, and other problems that have not been described will be clearly understood by those of ordinary skill in the art from the description below.

### Technical Solution

According to an aspect of the present invention, there is provided a method for enhancing an antibody-forming function using bio-informative energy light according to various embodiments of the present invention. The method includes administering a vaccine to a mammal and irradiating the vaccinated mammal with bio-informative energy light, wherein the bio-informative energy light has an intensity of 10⁻¹⁸ to 10⁻¹³ W/cm².

In an alternative embodiment, the bio-informative energy light may be applied in the evening.

In an alternative embodiment, the bio-informative energy light may be irradiated for at least two hours on a 24-hour basis.

In an alternative embodiment, the bio-informative energy light may promote the production of antibodies against the vaccine.

In an alternative embodiment, the bio-informative energy light may promote the production of antibodies against the vaccine and inhibit cytokine release.

In an alternative embodiment, the bio-informative energy light is provided by a bio-informative energy transmitter, which may include a light source generating light, a housing which includes an internal space and performs dispersion and diffuse reflection of the light entering the internal space, a first filter converting the dispersed and diffusely reflected light to monochromatic light, and a second filter diffracting and interfering with the converted light.

In an alternative embodiment, the housing may include a wall prism included in the internal space and dispersing and diffusely reflecting the entering light in multiple directions, and the dispersed and diffusely reflected light may be applied to the housing to emit photoelectrons in the internal space.

In an alternative embodiment, the inner wall of the housing may be formed of stainless-steel, the wall prism may be formed of an acrylic material and supported by the inner surface, and the second filter may cause continuous diffraction and interference through a plurality of prism disks to modulate the converted light.

In an alternative embodiment, the bio-informative energy transmitter may further include a third filter which filters the light transmitted from the second filter. The third filter may be formed of a black body acrylic plate and filter light of a predetermined energy intensity of the light transmitted from the second filter to emit the filtered light to the outside.

Other specific details of the present invention are included in the detailed description and drawings.

### Advantageous Effects

According to various embodiments of the present invention, an effect of enhancing the antibody-forming function can be provided by utilizing bio-informative energy light.

Additionally, an effect of preventing a cytokine storm in the process of promoting the antibody-forming function can be provided.

The effects of the present invention are not limited to those mentioned above, and other effects that have not been described will be clearly understood by those of ordinary skill in the art from the description below.

### DESCRIPTION OF DRAWINGS

Various aspects are now described with reference to the drawings, where like reference numerals are used to refer generally to like components. In the following embodiments, for explanatory purposes, numerous specific details are set forth to provide a comprehensive understanding of one or more aspects. However, it will be apparent that such aspects can be implemented without these specific details.

FIG. 1 shows an exemplary flowchart related to a method for enhancing an antibody-forming function using bio-informative energy light according to an embodiment of the present invention.

FIG. 2 shows a photograph of a bio-informative energy irradiation device installed in pigsty for an experiment according to an embodiment of the present invention.

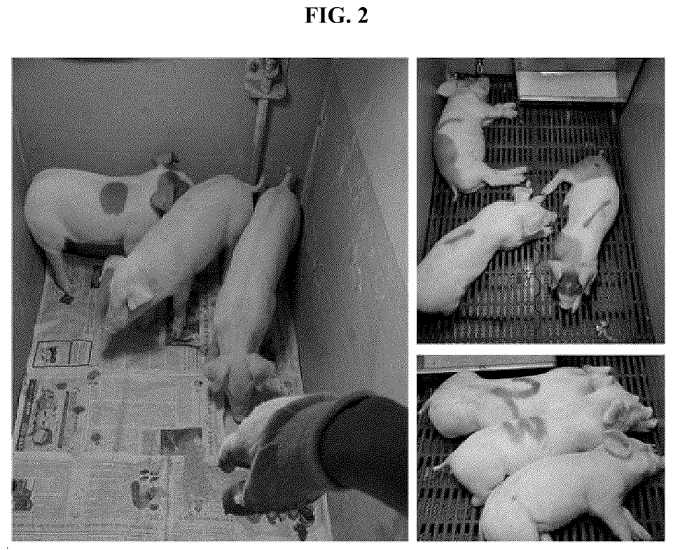

FIG. 3 shows an experimental result showing the relative abundance of metabolites in the serum of each experimental group and control group according to an embodiment of the present invention.

FIG. 4 shows an experimental result measuring the change in mitochondrial activation when bio-informative energy light is applied in the morning and evening according to an embodiment of the present invention.

FIG. 5 shows an experimental result measuring the change in mitochondrial activation when bio-informative energy light is or is not applied in the evening according to an embodiment of the present invention.

FIG. 6 is an exemplary diagram illustrating a cross-sectional view of a bio-informative energy transmitter according to an embodiment of the present invention.

### BEST MODE OF THE INVENTION

Various embodiments and/or aspects are disclosed with reference to the drawings below. In the following description, for purposes of explanation, numerous specific details are set forth to aid in the overall understanding of one or more aspects. However, it will also be apparent to one of ordinary skill in the art that these aspects may be practiced without these specific details. The following description and the accompanying drawings describe in detail certain exemplary aspects of one or more aspects. However, these aspects are examples, and some of the various methods in the principles of various aspects may be used, and the descriptions are intended to be inclusive of all aspects and their equivalents. Specifically, the terms “embodiment,” “example,” “aspect,” and “illustration” used herein should not be construed to imply that any aspect or design is better or more advantageous than other aspects or designs.

Hereinafter, identical or similar components, regardless of drawing symbols, will be given the same reference number and duplicate descriptions thereof are omitted. In addition, in description of embodiments disclosed in the specification, when it is determined that such detailed description of the known art would obscure the essence of embodiments disclosed herein, such detailed description is omitted. In addition, the accompanying drawings are intended only to facilitate easy understanding of the embodiments disclosed in the specification, and the technical ideas disclosed in the specification are not limited by the accompanying drawings.

Although the terms “first,” “second,” etc., are used to described various elements or components, these elements or components are not limited by these terms. These terms are used only to distinguish one element or component from another. Therefore, it is to be understood that a first element or component mentioned below may be a second element or component within the technical concept of the present invention.

Unless defined otherwise, all terms (including technical and scientific terms) used herein may be used in a sense that is commonly understood by one of ordinary skill in the art to which the present disclosure belongs. Also, generally used terms defined in dictionaries are not ideally or excessively interpreted unless explicitly defined otherwise.

Additionally, the term “or” is indented to mean an inclusive “or” rather than exclusive “or.” That is, unless otherwise specified or clear from the context, “X uses A or B” is intended to mean one of the natural inclusive substitutions. That is, “X uses A or B” can apply to any of the cases in which X uses A; X uses B; or X uses both A and B. In addition, the term “and/or” used herein should be understood to refer to and include all possible combinations of one or more of the related items listed.

In addition, it should be understood that the terms “comprise/include” and/or “comprising/including” indicate the presence of the features and/or components, but do not preclude the presence or addition of one or more other features, components, and/or groups thereof. In addition, unless otherwise specified or clear from the context to refer to the singular form, the singular should generally be construed in this specification and claims to mean “one or more.”

When a first component is mentioned to be “connected to” or “in contact with” a second component, it will be understood that the first component may be directly connected to or in contact with the second component, or a third component may be interposed therebetween. On the other hand, when a first component is mentioned to be “directly connected to” or “in direct contact with” a second component, it will be understood that there is no other component therebetween.

The purpose and effects of the present invention, and the technical configurations for achieving them will become clear with reference to the embodiments described in detail below together with the accompanying drawings. In explaining the present invention, when it is judged that detailed description of a known function or configuration may unnecessarily obscure the gist of the present invention, the detailed description will be omitted. In addition, the terms described below are terms defined in consideration of the functions within the present invention, and these may vary depending on the intention or custom of a user or operator.

However, the present invention is not limited to the embodiments disclosed below and may be implemented in a variety of different forms. These embodiments are provided solely to make the disclosure of the present invention complete and to fully inform those skilled in the art of the scope of the present invention, and the present invention is defined only by the scope of the claims. Therefore, definitions should be based on the contents throughout this specification.

In order for the cells that make up a living organism to function normally, they need energy that provides vitality. Photon energy from light stimulates the atoms that make up the cells, changing low-level electron energy surrounding the atoms into high-level electron energy. Electrons that have been converted to high-level energy in a very short period of time return to low-level energy, and when this happens, photons that have been absorbed are emitted, generating resonance energy. This resonance energy is called biophotons or bio-energy, and plays a role in promoting the metabolic functions of a living organism. A bio-informative energy light projector of the present invention may be a device invented based on the theory that the principles of the interrelationship between light, cells, and resonance energy affect a living organism.

Meanwhile, today, highly contagious viruses such as influenza viruses and novel coronaviruses are the cause of various diseases and a threat to human life. Viruses are so small that they can easily spread through animal feces, water, air, etc. depending on the type. When a contagious virus appears in a densely populated area, it can spread throughout a city in an instant. The development of vaccines is essential to prevent the spread of such viruses. A vaccine is made by weakening the power of viruses that cause a disease or by using a part of the viruses, and when it is administered to a person, antibodies that have an immune effect are produced.

However, there is concern that even vaccinated users may continue to be exposed to various viral infections due to insufficient antibody production, and there is concern that vaccination may cause side effects.

The present invention may provide a method for promoting the antibody-forming function of a vaccinated mammal using bio-informative energy light, and minimizing side effects caused by vaccination.

Bio-informative energy light is a type of light that is useful to living organisms. When radiated onto a living organism, bio-informative energy light can have positive effects on the living organism, such as activating metabolism, enhancing immunity, and enhancing cell growth. Bio-informative energy light may be polychromatic, have coherence and a visible range, and be polarized.

When such bio-informative energy light has a specific intensity and wavelength and is radiated at a certain distance from a mammal at a specific time zone, it may maximize the antibody-forming function of the vaccinated mammal. Specific characteristics of bio-informative energy light that maximize the antibody-forming function of the vaccinated mammal and experimental results showing that the antibody-forming function is improved through the corresponding bio-informative energy light will be described with reference to the drawings and tables below.

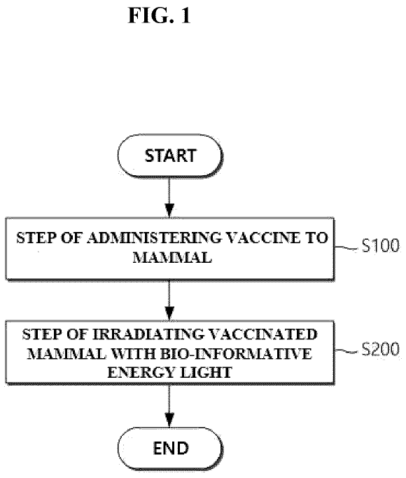

FIG. 1 shows an exemplary flowchart related to a method for enhancing an antibody-forming function using bio-informative energy light according to an embodiment of the present invention. The order of the steps illustrated in FIG. 1 may be changed as needed, and at least one step may be omitted or added. That is, the steps illustrated in FIG. 1 are merely an embodiment of the present invention, and the scope of the present invention is not limited thereto.

According to one embodiment of the present invention, the method for enhancing an antibody-forming function using bio-informative energy light may include administering a vaccine to a mammal (S100). In one embodiment, the vaccine of the present invention may be for producing antibodies for immunity against a specific virus. For example, the vaccine may be a porcine coronavirus vaccine, which, when administered to a pig, produces antibodies against the corresponding virus. The specific description of the vaccine and the vaccinated mammalian species is only exemplary, and the present invention is not limited thereto.

In one embodiment, the mammal may be, for example, at least one selected from a pig, a goat, sheep, a cow, an ox, a horse, a deer, a roe deer, a dog, a cat, a Bactrian camel, a rhinoceros, a hippopotamus, a giraffe, an elephant, a bear, a tiger, a lion, a leopard, a hyena, a badger, a fox, a wolf, a weasel, a rat, a squirrel, a hamster, a guinea pig, a beaver, a rabbit, a koala, a kangaroo, a monkey, a chimpanzee, and an orangutan, but the present invention is not limited thereto.

According to an embodiment of the present invention, the method for enhancing the antibody-forming function using bio-informative energy light may include applying bio-informative energy light to the vaccinated mammal (S200).

According to an embodiment, the bio-informative energy light may promote the production of antibodies against the vaccine. Specifically, when the vaccinated mammal is irradiated with the bio-informative energy light, the function of forming antibodies against the corresponding vaccine may be improved. For example, when a pig is irradiated with specific bio-informative energy light for a certain period of time after the porcine coronavirus vaccine is administered to the pig, the function of producing antibodies against porcine coronavirus may be further enhanced. In other words, simply by supplying bio-informative energy light after vaccination, the vaccination efficiency of a mammal, i.e., antibody production efficiency, may be further maximized. To maximize the antibody production efficiency, bio-informative energy light may have a predetermined wavelength, intensity, irradiation distance, irradiation cycle, or minimum irradiation time.

According to an embodiment, the bio-informative energy light may have a wavelength ranging from 300 to 870 nm. In a specific embodiment, the bio-informative energy light may have a wavelength ranging from 500 to 780 nm, a peak wavelength of 704.47 nm, a centroid wavelength of 676.10 nm, and a dominant wavelength of 588.45 nm.

In addition, according to an embodiment, the bio-informative energy light may have an intensity of 10⁻¹⁸ to 10⁻¹³ W/cm², and more preferably, 10⁻¹⁵ to 10⁻¹³ W/cm². The bio-informative energy light may include any type of light source without limitation, as long as it can satisfy the above-described intensity and can be radiated for a long period of time without side effects, and preferably includes a laser or LE light source used in phototherapy.

According to an embodiment, the bio-informative energy light may be applied in the evening. For example, the bio-informative energy light may be applied to the vaccinated mammal, and when the bio-informative energy light is applied, the antibody-forming function of the corresponding mammal may be enhanced. Specifically, when the bio-informative energy light is administered in the evening when the vaccinated mammal is inactive, the antibody-forming function may be maximized. That is, the bio-informative energy light may provide an antibody-enhancing effect when applied to a mammal in the evening (i.e., at night) when it is inactive.

In addition, in an embodiment, the bio-informative energy light may be irradiated for at least two hours. Specifically, when the bio-informative energy light is irradiated for at least two hours per day (i.e., on a 24-hour basis), the antibody-forming function of a mammal can be enhanced. For example, when the bio-informative energy light is applied to a vaccinated mammal for less than 2 hours per day, the antibody-forming function of the corresponding mammal does not go beyond the general range. In other words, the bio-informative energy light can help enhance the antibody production in mammals when applied continuously for at least two hours on a 24-hour basis. In one embodiment, the bio-informative energy light can cause significant improvement in the antibody-forming function of mammals when applied for at least 2 hours per day for at least 28 days.

In one embodiment, the bio-informative energy light may promote the production of antibodies against the vaccine, and inhibit cytokine release. Cytokines are proteins secreted from immune cells and act as immune regulators by binding to specific receptors in autocrine signaling, paracrine signaling, and endocrine signaling. There are various types of cytokines involved in cell proliferation, differentiation, apoptosis and wound healing, and many of them are particularly involved in immunity and inflammation. Cytokines have different effects depending on the state of target cells. It has been reported that a single cytokine exhibits opposing functions of immunostimulation and immunosuppression in immune responses. Cytokines regulate the expression of other cytokines, thereby initiating a cytokine cascade. Cytokine-producing cells interact with cytokines in a series of cascades, forming a complex cytokine network. A cytokine storm, in which cytokines are overproduced, is a cause of death. Cytokines are produced as a defense mechanism of the immune system against infection, but when excessive reaction occurs, it can cause airway obstruction or multiple organ failure. Since an active immune response produces excessive amounts of cytokines, excessive amounts of cytokines may be toxic to young, healthy mammals. That is, although cytokines may enhance immune responses, excessive cytokines may actually have adverse effects on mammals.

The bio-informative energy light of the present invention, when applied to a vaccinated mammal, may enhance antibody production for immunity and inhibit cytokine release. That is, the bio-informative energy light of the present invention may provide the effects of maximizing the antibody production efficiency caused by vaccination and preventing the risk caused by a cytokine storm.

According to an embodiment of the present invention, the maximization of the antibody-forming function to increase the immunity of a vaccinated mammal by the bio-informative energy light may be confirmed by the following experimental procedures and results thereof.

The following experiments were conducted to confirm the effects of bio-informative energy light on the growth capacity, immune system, and metabolism of mammals.

In one embodiment, the experiments were conducted on a total of 30 pigs, each of which was 21 days old and weighed an average of 7.06±0.11 kg, and divided into an experimental group (i.e., an experimental group irradiated with bio-informative energy light) and a control.

The experiments were performed in metal cages with plastic floors (1.2 m×2.4 m), and the average temperature of the cages was maintained between 25° C. and 30° C., and the humidity thereof was maintained between 61% and 66%.

The experiments were conducted for 48 days, and values measured from the experimental group and control were recorded at each of 14, 24, and 48 days after vaccination. Here, the experimental group may refer to pigs irradiated with the bio-informative energy light of the present invention for 2 hours or more per day.

Here, because the intensity of the bio-informative energy light is too weak to be measured using a spectrometer, the intensity value was measured 2 cm in front of the end of the light irradiation device. Meanwhile, since the intensity of light is attenuated inversely proportional to (distance) 2, when installed in an actual pigsty, the light irradiation device was installed at a radius of approximately 2 m from the mammal, and the intensity of the final light source was confirmed to be 1×10⁻¹⁵ to 10⁻¹³ W/cm².

TABLE 1
Growth ability

| Item | Control | Experimental group | P-value |
|---|---|---|---|
| Initial BW (kg) | 7.07 | 7.05 | 0.992 |
| Final BW (kg) | 31.7 | 34.17 | 0.08 |
| d 14 | | | |
| ADG (g) | 371 | 395 | 0.204 |
| ADFI (g) | 512 | 522 | 0.447 |
| G:F | 0.725 | 0.756 | 0.27 |
| d 28 | | | |
| ADG (g) | 455 | 521 | 0.065 |
| ADFI (g) | 679 | 736 | 0.121 |
| G:F | 0.67^{b} | 0.71^{a} | 0.039 |
| d 48 | | | |
| ADG (g) | 653 | 715 | 0.178 |
| ADFI (g) | 1.145 | 1.181 | 0.622 |
| G:F | 0.57^{b} | 0.61^{a} | 0.018 |
| Overall | | | |
| ADG (g) | 513 | 565 | 0.088 |
| ADFI (g) | 825 | 859 | 0.28 |
| G:F | 0.62 | 0.66 | 0.113 |

Referring to Table 1, in the experimental group irradiated with the bio-informative energy light, it can be confirmed that the initial body weight (initial BW) increased more than that in the control group not irradiated with the bio-informative energy light. Specifically, it was confirmed that, in the experimental group (i.e., the group irradiated with bio-informative energy light), the average initial BW of 15 pigs was 7.07 kg, but after 48 days, the weight increased by 27.1 kg to 34.17 kg, and in the control (i.e., the group not irradiated with bio-informative energy light), the average initial BW of 15 pigs was 7.05 kg, and after 48 days, the weight increased by 24.63 kg to 31.7 kg. That is, in the experimental group irradiated with the bio-informative energy light, it can be confirmed that the weight increased by 2.47 kg compared to the control irradiated with the bio-informative energy light.

Particularly, it can be confirmed that the measurement values of average daily feed intake (ADFI) and average daily gain (ADG) were higher in the experimental group than in the control on days 14, 24, and 48.

In addition, in the experimental group, it can be confirmed that the gain-to-feed ratio (G:F) was consistently higher than the control, and the p-value (reliability value of the corresponding information) of 0.05 or less confirms that this is very reliable information.

That is, like the above-described experimental results, in the pigs irradiated with the bio-informative energy light, it was confirmed that the total weight gain was significantly increased as the ADFI, ADG, and G:F were improved compared to the pigs not irradiated with the light. In other words, it can be confirmed that the growth ability of the mammals irradiated with the bio-informative energy light of a specific intensity and wavelength for at least two hours per day is improved.

In addition, blood samples were collected using Becton Dickinson anticoagulant-free disposable vacuum tubes from the experimental group and the control. Serum samples were centrifuged for 15 minutes and stored at −20° C., and then each sample was analyzed. The analysis was performed using the Hematology System (Drew Scientific, Oxford, CT). As a result of analysis, the measurement values of white blood cells (WBC), lymphocytes, red blood cells (RBC), and the concentration of WBCs containing neutrophils, monocytes, eosinophils, and basophils were obtained. By using the ELISA kit, the measurement values of immunoglobin G (IgG), immunoglobin A (IgA), IL-1β, TNF-α, and IL-6 were obtained.

TABLE 2
Blood analysis

| Item | Control | Experimental group | P-value |
|---|---|---|---|
| d 14 | | | |
| WBC | 25.82 | 24.86 | 0.166 |
| Lymphocytes | 61.7 | 62.47 | 0.395 |
| Neutrophils | 32.7 | 34.57 | 0.153 |
| Monocytes | 6.4 | 5.53 | 0.523 |
| Eosinophils | 1.56 | 1.53 | 0.778 |
| Basophils | 0.11 | 0.1 | 0.869 |
| d 28 | | | |
| WBC | 24.91 | 26.01 | 0.614 |
| Lymphocytes | 63.57 | 64.5 | 0.074 |
| Neutrophils | 36.07 | 37.5 | 0.186 |
| Monocytes | 5.6 | 5.7 | 0.910 |
| Eosinophils | 1.9 | 1.73 | 0.189 |
| Basophils | 0.28 | 0.26 | 0.856 |

Referring to Table 2, in the serum analysis results, there were no significant differences in WBCs, lymphocytes, neutrophils, monocytes, eosinophils or basophils in the blood from those of the control.

TABLE 3
Blood analysis

| Item | Control | Experimental Group | P-value |
|---|---|---|---|
| d 28 | | | |
| IgA (ng/ml) | 8.72^{b} | 10.28^{a} | 0.001 |
| IgG (ng/ml) | 22.59^{b} | 26.12^{a} | 0.001 |
| d 48 | | | |
| IgA (ng/ml) | 33.76^{b} | 58.41^{a} | <0.001 |
| IgG (ng/ml) | 41.56^{b} | 45.15^{a} | 0.036 |

An immunoglobulin is a glycoprotein molecule that is produced as an immune response by antigen stimulation, and mainly binds specifically to a specific antigen in the blood, causing an antigen-antibody reaction. In one embodiment, immunoglobulins, also known as antibodies, are produced from B lymphocytes and serve to remove antigens from pathogenic microorganisms such as bacteria and viruses through precipitation or agglutination. In addition, immunoglobulins induce various immune functions by interacting with other elements of the immune system. That is, a higher level of immunoglobulin may mean improved immune function.

Referring to Table 3, it can be confirmed that both immunoglobulin A (IgA) and immunoglobulin G (IgG) have high levels in the experimental group irradiated with the bio-informative energy light. That is, it was able to be confirmed that the immune function of the experimental group irradiated with the bio-informative energy light for at least 2 hours per day for 48 days was significantly improved. Particularly, the p-value (confidence value of the corresponding information) corresponding to each group is 0.05 or less, confirming that the information is very reliable.

In an additional example, the same blood analysis was performed on pigs irradiated with the bio-informative energy light for only one hour (i.e., 60 minutes) per day, i.e., on a 24-hour basis, as a control and pigs irradiated with the bio-informative energy light for two hours (i.e., 120 minutes) per day on a 24-hour basis as an experimental group. The results are shown in Table 4 below.

TABLE 4
Blood analysis

| Item | Control | Experimental group | P-value |
|---|---|---|---|
| d 28 | | | |
| IgA (ng/ml) | 9.72^{b} | 9.32^{a} | 0.001 |
| IgG (ng/ml) | 22.31^{b} | 22.94^{a} | 0.001 |
| d 48 | | | |
| IgA (ng/ml) | 35.27^{b} | 36.42^{a} | <0.001 |
| IgG (ng/ml) | 42.36^{b} | 43.16^{a} | 0.032 |

Referring to Table 4, it can be confirmed that there is little difference between the IgA and IgG levels corresponding to the experimental group and the control. In other words, in the case of pigs irradiated with the bio-informative energy light for one hour on a 24-hour basis (i.e., per day), it was able to be confirmed that there was no significant difference from the group not irradiated with the bio-informative energy light. This may mean that immune function was not improved.

Based on the content of Tables 3 and 4, it can be confirmed that, in order to achieve a significant increase in immunoglobulins, the bio-informative energy light must be supplied to mammals for at least two hours on a 24-hour basis. In other words, the supply of the bio-informative energy light to mammals for at least two hours on a 24-hour basis may be a functional feature essential to enhance the immune function of the corresponding mammal.

TABLE 5
Blood analysis

| Item | Control | Experimental group | P-value |
|---|---|---|---|
| d 28 | | | |
| TNF-α (pg/ml) | 238.8 | 224 | 0.628 |
| IL-1β (pg/ml) | 17.17 | 14.33 | 0.263 |
| IL-6 (pg/ml) | 178.5^{a} | 170.5^{b} | 0.023 |
| d 48 | | | |
| TNF-α (pg/ml) | 473.6^{a} | 427.9^{b} | 0.043 |
| IL-1β (pg/ml) | 186.0^{a} | 155.4^{b} | 0.005 |
| IL-6 (pg/ml) | 323.0^{a} | 289.5^{b} | 0.02 |

In addition, as shown by the result values in Table 5, the blood analysis can show that, on both days 28 and 48, cytokine levels in the experimental group were lower than those in the control. Tumor necrosis factor-α (TNF-α) is a cytokine that regulates immune cells and is mainly secreted by macrophages, IL-1β is a cytokine that regulates inflammation and immune responses to infection, and IL-6 is a cytokine that is secreted by various cells such as T lymphocytes and macrophages and promotes immune responses.

Specifically, it can be confirmed that the levels of TNF-α, IL-1β, and IL-6 in the experimental group are higher than those of TNF-α, IL-1β, and IL-6 in the control. Particularly, it can be confirmed that, on day 28, the differences in TNF-α, IL-1β, and IL-6 corresponding to each of the experimental group and the control were approximately 14.8, 2.84, and 8, respectively, but on day 48, the differences in cytokine level corresponding thereto were 46, 30.6, and 33.5, respectively, showing a significant difference.

That is, it was confirmed that cytokine release was inhibited in the experimental group irradiated with the bio-informative energy light for at least 2 hours per day for 48 days.

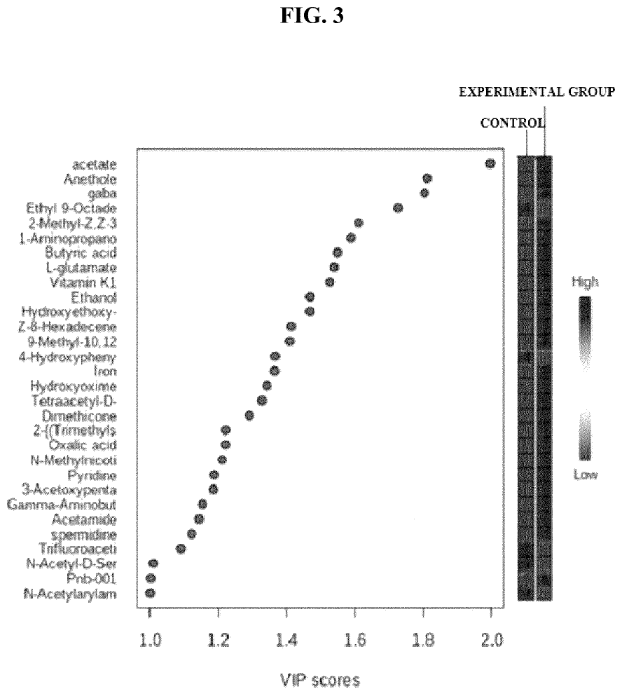

Additionally, to analyze metabolic profiles associated with the irradiation with bio-informative energy light, the relative abundance of metabolites in serum was assessed by comprehensive metabolomic analysis using gas chromatography-mass spectrometry (GC-MS). The corresponding result values are shown in FIG. 3.

As shown in FIG. 3, the results of the above-described experiments showed that the differences between the control and the experimental group can be discerned. More specifically, it can be confirmed that the concentrations of acetate, anethole, GABA, 2-methyl-Z,Z-3,13-octadecadienol, 1-aminopropanol-(2), butyric acid, L-glutamate, vitamin K1, ethanol, iron, hydroxyoxime, tetraacetyl-D-xylonic nitrile, dimethicone, 2-[(trimethylsilyl)oxy]tetradecanoic acid, oxalic acid, N-methylnicotinamide, pyridine, 3-acetoxypentadecane, gamma-aminobutyric acid, acetamide, spermidine, and Pnb-001 were increased in the photonia treatment, and the concentrations of ethyl 9-octadecenoate, 4-hydroxyphenylethanol, trifluoroacetic acid, N-acetyl-D-serine, and acetylarylamine were greater in the experimental group than in the control.

The dose and effectiveness of radiation on the metabolism of living tissue are assessed by multiparameter approaches for biomarker identification. Among these, metabolomics is a method of detecting a wide range of small molecules that reflect physiological conditions at the cellular level and provide valid indicators. Differences in daily weight gain between the experimental group and the control are associated with energy and protein metabolism.

It has been reported that increased anethole in blood is associated with anti-inflammatory responses, and shows high anti-inflammatory and antioxidant effects in acute lung injury induced by LPS. Anethole reduces the production of inflammatory cytokines such as TNF-α and IL-6, which is consistent with the results of this experiment.

Additionally, γ-aminobutyric acid (GABA) is well known as an extracellular signaling molecule produced mainly in the brain, blood, and pancreatic islets, and secreted from pancreatic islet B cells. GABA is a major inhibitory neurotransmitter that suppresses the production of 47 types of cytokines, including IL-1β, IFN-γ, TNF-α, IL-6, and IL-12, through the regulation of human differentiated 4+ T cells. The next step is to cluster the analyzed results to identify metabolic pathways. By the application of bio-informative energy light, the metabolic pathways of six amino acids, including glutamate, phenylalanine, alanine, and aspartate, were affected, and it is thought that these pathways are involved in growth and protection against inflammatory responses in pigs. When the availability of amino acids increases, protein biosynthesis increases. Glutamine is a very important metabolite in D-glutamine, D-glutamate, alanine, and aspartate metabolic pathways, and was detected at a high frequency in pigs irradiated with bio-informative energy light. Glutamine is one of the most abundant elements in protein synthesis and may be the cause of increased daily weight gain in pigs after the irradiation with bio-informative energy light. GABA production in the body induces D-glutamate production by GABA transaminase activity in the mitochondria, and when the level of glutamine in the amino acid pool in the body is high, an immune response-regulating effect and the inflammatory response of macrophages increase. Inflammatory response is a symptom that appears in pigs when inflammatory cytokines such as TNF-α, IL-1β, and IL-6 are secreted. The decrease in inflammatory cytokines in the experimental group may be mainly caused by an increase in the D-glutamate metabolic pathway.

D-glutamine and D-glutamate metabolisms are known to reduce oxidative stress and damage caused by free radicals in the kidney. In addition, glutamine and alanine serve as sources of glucose when the energy balance for glucose synthesis in the body is not met. Phenylalanine is an essential amino acid and a substrate for the neurotransmitter tyrosine, which is known to be an important factor in immunity and energy metabolism. Phenylalanine metabolism was increased in the experimental group, and the increase in the activity of an amino acid metabolic pathway serves as a major factor in improving the weight gain and feed efficiency, and immunity in pigs.

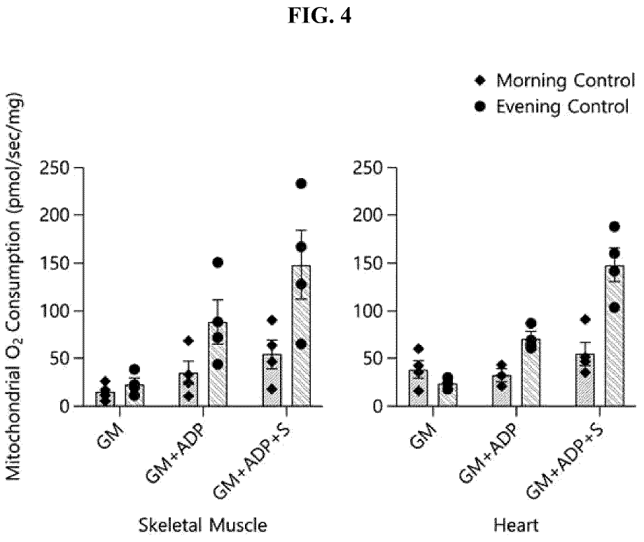

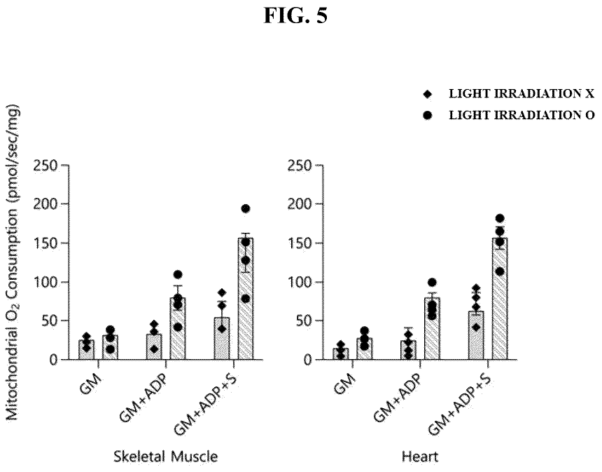

As described above, when bio-informative energy light having a wavelength of 500 to 780 nm and an intensity of 10⁻¹⁵ to 10⁻¹³ W/cm² is applied to mammals administered a vaccine for a minimum of 2 hours for at least 28 days on a 24-hour basis (i.e., per day), the growth ability and immune function (i.e., antibody-forming function) of the corresponding mammals may be improved. Particularly, in the process of improving immune function, cytokine release is suppressed, and thus the effect of preventing the risk caused by a cytokine storm can be provided. According to an embodiment, the bio-informative energy light may be applied during the night time. Here, the night time may be from 7:00 PM to 7:00 AM. In one embodiment, to enhance the antibody-forming function of vaccinated mammals, the bio-informative energy light may be applied to mammals in the evening. When the bio-informative energy light is applied to mammals in the evening rather than in the morning, the antibody-forming function may be maximized. Hereinafter, referring to FIGS. 4 and 5, the changes in mammalian immune function when bio-informative energy light is applied in the morning and evening and when it is not applied will be described.

The experiment related to FIG. 4 was conducted by forming an experimental group and a control with a total of 12 mice. Specifically, six mice were grouped as a morning control (e.g., a control), and the other six mice were grouped as an evening control (e.g., an experimental group). Bio-informative energy light was applied in a 12-hour cycle (e.g., 7:00 AM to 7:00 PM, or 7:00 PM to 7:00 AM) corresponding to the morning and evening, respectively, and the activation of mitochondrial functions in the heart and muscles was measured for each group. In one embodiment, it may be possible to make a quantitative measurement of how well the mitochondria are functioning based on how much oxygen is consumed.

In one embodiment, the mitochondria are one type of the cell organelles and can be involved in cellular respiration. For example, the more actively a cell respires, the more mitochondria it contains. Accordingly, a lot of energy can be produced. The mitochondria play a role in synthesizing ATP, which is an energy source, through food taken into the body. The inner membrane of the mitochondria contains a protein called ATP synthase, which can produce ATP. Hydrogen ions created between the inner membrane and the outer membrane of the mitochondria from food enter the inner membrane of the mitochondria, and a phosphate and ADP (the form in which two phosphates are combined with adenosine) are combined by the ATP synthase, forming ATP (the form in which three phosphates are combined with adenosine). In addition, the mitochondria play a role in preventing cells that have already lost their function from transforming into cancer cells or other cells by absorbing cells with damaged DNA or by destroying the cells that have lost their function. The mitochondria may play a major role in immunity. For example, when a virus enters a cell, immune cells may begin to work actively through the mitochondrial action. That is, when the mitochondria work actively and produce a lot of energy, immune cells also actively act, which can provide an effect of enhancing immunity.

Referring to FIG. 4, it can be confirmed that the mitochondrial oxygen consumption corresponding to the experimental group irradiated with bio-informative energy light in the evening group is significantly higher than that of the experimental group irradiated with bio-informative energy light in the morning group. In one embodiment, it can be confirmed that the more types of metabolic products (or metabolites) occur during the mitochondrial metabolism, the greater the difference in mitochondrial oxygen consumption between the evening group and the morning group.

That is, it can be confirmed that the mitochondrial function of the heart and muscles of the experimental group irradiated with bio-informative energy light for 12 hours in the evening is more activated than the control group irradiated with bio-informative energy light for 12 hours in the morning. This is the experimental result showing that immune enhancement is further maximized when bio-informative energy light is applied in the evening. That is, the present invention can maximize the enhancement of immunity by applying bio-informative energy light to a vaccinated mammal in the evening for the enhancement of immunity.

In addition, according to an embodiment, the experiment related to FIG. 5 was conducted on an experimental group and a control with a total of 12 mice. Specifically, six mice in a first group (i.e., the experimental group) were irradiated with bio-informative energy light in the evening (e.g., 7:00 PM to 7:00 AM), and the other six mice in a second group (i.e., the control) were not irradiated with bio-informative energy light, and then the activation of mitochondrial function was measured for each group.

Referring to FIG. 5, in the second group not irradiated with bio-informative energy light in the evening, it can be confirmed that the oxygen consumption of the mitochondria is significantly lower compared to the first group irradiated with bio-informative energy light in the evening. Here, the oxygen consumption of the mitochondria may be related to the degree of activation of the mitochondria. In one embodiment, it may be possible to make a quantitative measurement of how active the mitochondria are based on how much oxygen was consumed. That is, when bio-informative energy light is applied in the evening (e.g., 7:00 PM to 7:00 AM), it can be confirmed that the activity information of the mitochondria is maximized compared to when bio-informative energy light is not applied in the evening. In one embodiment, since the activation of the mitochondria causes the enhancement of immunity, it can be clearly inferred from the experimental results described above that the experimental group irradiated with bio-informative energy light in the evening has increased efficacy of enhancing immunity compared to the control not irradiated with bio-informative energy light. The above-described experiments shown in FIGS. 4 and 5 were conducted using mice among various mammals, but it was confirmed that similar effects occur in species other than mice, such as sows or dairy cows.

In summary, the experimental results above show that the irradiating the mammals with bio-informative energy light is excellent in the enhancement of immunity than not irradiating them with bio-informative energy light. Particularly, it can be confirmed that the irradiation of mammals with bio-informative energy light in the evening rather than the morning can maximize the enhancement of immunity.

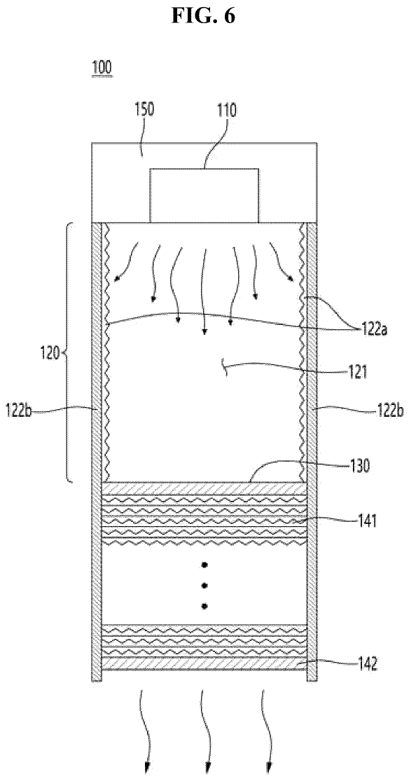

In one embodiment of the present invention, the bio-informative energy light described above may be supplied by a bio-informative energy transmitter. Specifically, as shown in FIG. 6, the light emitted from the bio-informative energy transmitter 100 may be applied to a mammal.

FIG. 6 is an exemplary diagram illustrating a cross-sectional view of a bio-informative energy transmitter that emits bio-informative energy light according to an embodiment of the present invention. As shown in FIG. 6, the bio-informative energy transmitter 100 may include a light source 110, a housing 120, a first filter 130, a second filter 141, a third filter 142, and a heat dissipation member 150. The above-described components are exemplary, and the scope of the present invention is not limited by the above-described components. That is, depending on the aspects of the embodiments of the present invention, additional components may be included, or some of the components described above may be omitted.

According to an embodiment of the present invention, the bio-informative energy transmitter 100 may include a light source 110. The light source 110 may be a radiator that emits infrared light, visible light, or ultraviolet light. The light source 110 may directly or indirectly convert thermal or electric energy into radiation energy. The light source 110 may generate light such as infrared light, visible light, and ultraviolet light, for example, through combustion-induced light emission, discharge-induced light emission, or semiconductor emission. The specific description of the above-described method for generating light is merely exemplary, and the present invention is not limited thereto. The light source 110 may be disposed in one direction of the housing 120 and transmit light to the housing 120. In one embodiment, the light source 110 may consist of a plurality of LED elements. Each of the plurality of LED elements may be a semiconductor element that generates and provides light through current.

According to one embodiment of the present invention, the bio-informative energy transmitter 100 may include a heat dissipation member 150. In one region of the heat dissipation member 150, a power source that applies electric power to the light source 110 may be included. The heat dissipation member 150 may diffuse heat generated from the power source. That is, the heat dissipation member 150 may effectively control the increase in heating value generated within the electronic device in the course of ongoing use, i.e., the exothermic phenomenon.

The heat dissipation member 150 may be formed of a material having excellent thermal conductivity. The higher the thermal conductivity, the better the thermal energy can be transferred (i.e., diffused) well to another place, effectively controlling heat generation. For example, the heat dissipation member 150 may be provided with a metal and ceramic material having high thermal conductivity. In addition, for example, the heat dissipation member 150 may be provided with a polymer composite material formed by using a carbon-based filler such as graphite, carbon fiber, carbon nanotubes, or graphene with excellent thermal conductivity, or a ceramic-based filler such as boron nitride, aluminum nitride, and alumina alone or in combination and uniformly dispersing and fully charging it on a polymer matrix. The specific description of the above-described material for the heat dissipation member is exemplary, and the present invention is not limited thereto. According to an additional embodiment, the heat dissipation member 150 may be provided with a material having a thermal expansion coefficient below a predetermined level, thereby reducing the possibility of failure due to component failure caused by heat generation.

The heat dissipation member 150 may be positioned in one direction (e.g., upward) from the light source 110, and may be included adjacent to the housing 120. As shown in FIG. 6, the heat dissipation member 150 is provided in contact with one surface of the housing 120, so that heat generated during the process of generating light by the light source 110 may be transferred to the housing 120. That is, the heat dissipation member 150 may diffuse the generated heat to the housing 120. In this case, the housing 120 may create an internal space 121 where a photoelectric effect or heat emission occurs. As the heat dissipation member 150 diffuses heat to the housing 120, the thermal electron emission efficiency is improved in the internal space 121 of the housing 120, resulting in maximizing the thermal electron emission and maximizing the efficiency of generating bio-informative energy light.

According to one embodiment of the present invention, the bio-informative energy transmitter 100 may include the housing 120. In the internal space 121 of the housing 120, the entering light may be dispersed and diffusely reflected in multiple directions. As shown in FIG. 6, in the internal space 121 of the housing 120, a wall prism 122a may be formed in the inner direction of the housing 120, and cause the dispersion and diffuse reflection of light, emitting photoelectrons to the internal space 121.

Specifically, the light generated from the light source 110 may be applied to the internal space 121 of the housing 120, and in the corresponding internal space 121, photoelectrons may be produced while light hits the wall. In this case, since the light itself emitted from the light source 110 is composed of photons with various levels of energy, the energy levels of the photoelectrons generated in the internal space 121 may also vary.

To explain in detail, light from the light source 110 may be dispersed and diffusely reflected through the wall prism 122a of the housing to emit photoelectrons. Specifically, the wall prism 122a may be composed of an acrylic material, and formed in the shape on a plane that is not parallel to the side surface of the housing 120. That is, the wall prism 122a may include a plurality of polygonal prisms protruding inward from the side wall of the housing 120 in a shape in which at least one pair of sides is non-parallel. For example, the plurality of polygonal prisms may be in the shape of triangle prisms. However, the shape of the plurality of polygonal prisms constituting the wall prism is not limited to the above shape, and they may be implemented in various shapes such as a polygonal pillar, a polygonal pyramid, a cone, or a sphere.

The multiple polygonal prisms that make up the wall prism 122a may be configured in various sizes from a few nanometers to a few millimeters. When the light emitted from the light source 110 is incident on the wall prism 122a (i.e., each of the plurality of polygonal prisms), the degree of refraction varies depending on a wavelength or frequency, which may cause dispersion. In other words, light is split into wavelengths (i.e., energy levels) through the wall prism 122a.

In addition, the housing 120 may support the wall prism 122a, and include an inner wall 122b made of a metal material. According to one embodiment, the inner wall 122b may be formed of a stainless-steel material. As shown in FIG. 6, the inner wall 122b may be formed along the inner surface of the cylindrical housing 120, and the wall prism 122a consisting of a plurality of polygonal prims may be formed using the inner wall 122b as a support. Accordingly, when the light generated from the light source 110 is applied to the housing 120, the light is transmitted to the inner wall 122b through the wall prism 122a.

The inner wall 122b may be formed of a metal material to restrain electrons. Specifically, within the inner wall 122b, electrons may be bound (or restrained) by the (+) charge of the nucleus and an electric force. The electrons restrained by the inner wall 122b may be emitted by light with various wavelengths. That is, as light is transmitted, photoelectrons may be emitted. In this case, because the light transmitted to the inner wall 122b is light dispersed into photons of various levels of energy through the wall prism 122a, the emission of photoelectrons may be maximized. That is, the photon absorption efficiency of the inner wall 122b may be increased through the wall prism 122a, and thus the emission of photoelectrons may be maximized. In this case, as the light itself emitted from the light source 110 is composed of photons with various levels of energy, the energy levels of photoelectrons generated in the internal space 121 may also vary.

According to an additional embodiment, the inner wall 122b may be formed of an aluminum (Al) material. When the inner wall 122b is formed of an aluminum material, the photoelectric emission efficiency may be further improved. Specifically, metals have their own unique work function (W) and threshold frequency. Here, the work function and threshold frequency may be the minimum energy and frequency of light that cause electrons restrained by the metal to be released, respectively. Aluminum may have a work function of 4.06 to 4.26 eV, which is lower than those of other metals. That is, when the inner wall 122b is made of an aluminum material, due to the low work function, the minimum energy of light for emitting photoelectrons may be reduced, and the photoelectrons may be emitted with low optical energy.

In addition, according to an embodiment, the work function may also be important in thermionic emission. Thermionic emission may mean that charge carriers flow from a surface over a potential energy barrier due to heat. Unlike the photoelectric effect, thermionic emission may use heat instead of photons to emit electrons. Specifically, the following equation is completed according to Richardson's law.

$$J = {AT}^{2} {e}^{\frac{- W}{kT}}$$

Here, J may be current density, T may be absolute temperature, W may be work function, K may be Boltzmann constant, and A may be Richardson's constant. That is, as the work function restraining electrons is lower, the efficiency of the thermionic emission may increase. Because aluminum has a work function of 4.06 to 4.26 eV, which is lower than that of other metals, the thermal energy required to emit thermionic electrons may be minimized, allowing the thermionic electrons to be emitted with relatively little thermal energy.

In other words, when the inner wall 122b is formed of an aluminum material, the photoelectric and thermionic emission efficiency may be improved. As a result, the increase in photoelectric and thermionic emission efficiency may contribute to the improvement in efficiency of generating bio-informative energy light.

According to one embodiment of the present invention, the bio-informative energy transmitter 100 may include a first filter 130. The first filter 130 may uniformly convert the dispersed and diffusely reflected light to monochromatic light and transmit it to the second filter 141.

More specifically, the first filter 130 may be formed of an acrylic material. For example, the first filter 130 may have an outer diameter corresponding to the inner diameter of the housing 120 and a thickness of 1 to 5 mm.

The first filter 130 may be connected to one end of the housing 120, and may transmit light from the internal space 121 of the housing 120. The light transmitted from the internal space 121 may be light that is dispersed and diffusely reflected through the wall prism 122a of the housing 120 (i.e., light from photoelectric emission or thermionic emission). Since the dispersed and diffusely reflected light has different characteristics of white light depending on light intensity and wavelength, non-uniform color distribution characteristics may be exhibited. Accordingly, the first filter 130 may convert the dispersed and diffusely reflected light into uniform monochromatic light. For example, the first filter 130 may convert the dispersed and diffusely reflected light (i.e., photoelectrons) to monochromatic light such as blue frequency energy. The first filter 130 may serve as a color correction filter for light.

That is, in the wall prism 122a of the housing 120, the dispersed and diffusely reflected light may convert into uniform monochromatic light in the process of passing through the first filter 130 and may be transmitted to a second filter 141 positioned in one direction (e.g., downward in FIG. 6) of the first filter 130. The first filter 130 serving as a color correction filter may convert light with various characteristics into uniform light with the same characteristics.

According to one embodiment of the present invention, the bio-informative energy transmitter 100 may include a second filter 141 formed by stacking a plurality of prism disks. In addition, the bio-informative energy transmitter 100 may include a third filter 142 that filters light transmitted from the second filter 141.

In one embodiment, the second filter 141 may modulate the converted light (i.e., light passing through the first filter) by continuous diffraction and interference using a plurality of prism disks. Specifically, as shown in FIG. 6, the second filter 141 may be implemented by stacking a plurality of prims discs.

The second filter 141 may be provided in contact with the first filter 130 in one direction (e.g., downward) by stacking several layers of prism disks. The light converted through the first filter 130 may cause continuous diffraction and interference while passing through each layer of the second filter 141, and thus can be modulated. Modulation of the converted light may mean, for example, modulation of light to have an optimal wavelength in order to enhance cell proliferation efficiency in a living organism. In a specific example, as the light is modulated through the second filter 141, the corresponding light may have a wavelength of 500 to 780 nm. Here, the light with a wavelength of 500 to 780 nm may be appropriate light for increasing the cell proliferation efficiency (e.g., improved fertility) of a living organism. In one embodiment, the second filter 141 may be characterized by modulating light to various wavelengths according to the pattern of arranging a plurality of prism disks. That is, the light passing through the second filter 141 may be modulated to an appropriate wavelength for enhancing an antibody-forming function in living organisms through continuous diffraction and interference in the process of passing through each layer (i.e., a plurality of prism disks).

In one embodiment, the third filter 142 may be formed of a black body acrylic plate. The black body acrylic material may serve as a filter that passes only light with a specific range of intensity. That is, the third filter 142 may allow only light with a certain range of intensity to be emitted to the outside through the black body acrylic material.

To elaborate, the third filter 142 may filter light with predetermined intensity of the light transmitted from the second filter 141, emitting the bio-informative energy light to the outside. Here, the predetermined intensity may mean the range of light related to the optimal intensity for enhancing the cell proliferation efficiency of living organisms. For example, the intensity of the light passing through the third filter 142 (i.e., bio-informative energy light) may be light ranging from 10⁻¹⁵ to 10⁻¹³ W/cm². In other words, the light with an intensity of 10⁻¹⁵ to 10⁻¹³ W/cm² may be light with the optimal intensity for enhancing the antibody-forming function of living organisms. For example, when light exceeding the range of 10⁻¹⁵ to 10⁻¹³ W/cm² (e.g., 10⁻¹¹) is applied to a living organism, it may not be the appropriate light (i.e., bio-informative energy light) for enhancing the antibody-forming function of the living organism.

That is, the third filter 142 may filter only light with a specific intensity of the light passing through the second filter 141 (e.g., light of a specific wavelength band) to be emitted to the outside. Accordingly, the light emitted to the outside may be bio-informative energy light, which is light with the optimal intensity for enhancing cell proliferation efficiency of a living organism.

According to one embodiment, the light generated from the light source 110 may be emitted to the outside sequentially through the internal space 121, the first filter 130, the second filter 141, and the third filter 142.

In summary, the heat dissipation member 150 may transfer (or diffuse) the heat generated in the process of generating light from the light source 110 to the internal space 121 of the housing 120, maximizing thermionic emission efficiency in the corresponding internal space 121. In addition, the light emitted from the light source 110 may be dispersed and diffusely reflected by the wall prism 122a, maximizing photoelectric emission efficiency. The light related to photoelectric and thermionic emission may pass through the first filter 130, and in this process, the dispersed and diffusely reflected light may be uniformly converted to monochromatic light. The light converted into uniform monochromatic light through the first filter 130 may be modulated to have a specific wavelength range through continuous diffraction and interference in the process of passing through the second filter 141 composed of a plurality of prism disks and thus transmitted to the third filter 142. The third filter 142 may allow only light having more than a certain energy intensity (i.e., bio-informative energy light) of the light transmitted through the second filter 141 to be emitted to the outside of the bio-informative energy transmitter 100.

That is, the bio-informative energy light that enhances cell proliferation efficiency in living organisms may be generated and emitted to the outside. Here, the bio-informative energy light may be light converted and modulated to have the optimal wavelength and intensity ranges for enhancing cell proliferation efficiency of living organisms in the process of passing through the second filter 141 and the third filter 142.

In addition, in the process of generating the bio-informative energy light, the heat dissipation member 150 may allow heat transfer to the internal space 121 to maximize thermionic emission efficiency, thereby improving the efficiency of generating the bio-informative energy light. In addition, in the process of generating bio energy light, photoelectric emission efficiency may be maximized by the wall prism 122a, resulting in improving the efficiency of generating bio-informative energy light.

That is, the bio-informative energy transmitter 100 of the present invention may generate bio-informative energy light having the optimal efficiency due to the structural characteristic of maximizing photoelectric emission and thermionic emission.

According to various embodiments of the present invention, an effect of enhancing an antibody-forming function using bio-informative energy light can be provided.

In addition, in the process of promoting the antibody-forming function, an effect of preventing a cytokine storm can be provided.

The effects of the present invention are not limited to the effects mentioned above, and other effects that have not been mentioned will be clearly understood by those of ordinary skill in the art from the description below.

It should be understood by those of ordinary skill in the art that, while the embodiments of the present invention have been described with reference to the accompanying drawings, the exemplary embodiments disclosed herein can be easily modified into other specific forms without departing from the technical spirit or essential features of the present invention. Therefore, the exemplary embodiments described above should be interpreted as illustrative in all aspects and not restrictive.

The specific implementations described in the present invention are exemplary and do not limit the scope of the present invention in any way. To simplify the specification, descriptions of conventional electronic components, control systems, software, and other functional aspects of the systems may be omitted. In addition, the connections or connecting members for lines between components shown in the drawings are illustrative of functional connections and/or physical or circuit connections, and may be represented in an actual device as alternative or additional various functional, physical or circuit connections. In addition, if there is no specific mention such as “essential,” “importantly,” etc., it may not be an essential component for the application of the present invention.

It is important to understand that the specific order or hierarchy of steps in the processes presented is an example of an exemplary approach. It should be understood that, based on design priorities, the specific order or hierarchy of steps in the processes within the scope of the present invention may be rearranged. The attached method claims provide elements of various steps in a sample order, but are not meant to be limited to the specific order or hierarchy presented.

The description of the presented embodiments is provided to enable those of ordinary skill in the art to use or implement the present invention. Various modifications to these embodiments will be apparent to those of ordinary skill in the art, and the general principles defined herein may be applied to other embodiments without departing from the scope of the present invention. Accordingly, the present invention is not limited to the embodiments presented herein, but should be construed in the broadest scope consistent with the principles and novel features presented herein.

[Description of numerals]

| | |
|---|---|
| 100: Bio-informative energy transmitter | 110: Light source |
| 110a: Plurality of LED elements | 120: Housing |
| 121: Internal space | 122a: Wall prism |
| 122b: Inner wall | 130: First filter |
| 141: Second filter | 142: Third filter |
| 150: Heat dissipation member | |

### Modes of the Invention

The relevant content has been described above in the best mode of the invention.

### INDUSTRIAL APPLICABILITY

The present invention can be utilized in the livestock farming or quarantine industry.

1. A method for enhancing an antibody-forming function using bio-informative energy light, comprising:
administering a vaccine to a mammal; and
irradiating the vaccinated mammal with bio-informative energy light;
wherein the bio-informative energy light has an intensity of 10⁻¹⁸ to 10⁻¹³ W/cm².

2. The method of claim 1, wherein the bio-informative energy light is irradiated in the evening.

3. The method of claim 1, wherein the bio-informative energy light is irradiated for at least two hours on a 24-hour basis.

4. The method of claim 1, wherein the bio-informative energy light promotes the production of antibodies against the vaccine.

5. The method of claim 4, wherein the bio-informative energy light promotes the production of antibodies against the vaccine, but inhibits cytokine release.

6. The method of claim 1, wherein the bio-informative energy light is provided by the bio-informative energy transmitter, and
the bio-informative energy transmitter includes a light source generating light; a housing including an internal space, and performing dispersion and diffuse reflection of the light entering the internal space; a first filter converting the dispersed and diffusely reflected light to monochromatic light; and a second filter diffracting and interfering with the converted light.

7. The method of claim 6, wherein the housing includes a wall prism included in the internal space and dispersing and diffusely reflecting the entering light in multiple directions, and the dispersed and diffusely reflected light is applied to the housing to emit photoelectrons in the internal space.

8. The method of claim 6, wherein the inner wall of the housing is formed of a stainless-steel material, the wall prism is formed of an acrylic material and supported by the inner surface, and the second filter modulates the converted light by causing continuous diffraction and interference through a plurality of prism disks.

9. The method of claim 6, wherein the bio-informative energy transmitter further includes a third filter filtering the light transmitted from the second filter; and the third filter is formed of a black body acrylic plate and filters light with a predetermined energy intensity of the light transmitted from the second filter to emit the filtered light to the outside.
